(19) United States
(12) Patent Application Publication
(10) Pub. No.: US 2017/0030821 A1
(43) Pub. Date: Feb. 2, 2017

(54) URINE SEDIMENT ANALYSIS WORKSTATION

(71) Applicant: Siemens Healthcare Diagnostics Inc., Tarrytown, NY (US)

(72) Inventor: Trevor Allison, Sible Hedingham (GB)

(73) Assignee: Siemens Healthcare Diagnostics Inc., Tarrytown, NY (US)

(21) Appl. No.: 15/303,862

(22) PCT Filed: Apr. 10, 2015
(86) PCT No.: PCT/US15/25379

§ 371 Date: Oct. 13, 2016

Related U.S. Application Data
(60) Provisional application No. 61/984,375, filed on Apr. 25, 2014.

(51) Int. Cl. G01N 15/14; G02B 21/36; G01N 33/493

(52) U.S. Cl. CPC G01N 15/1429; G01N 33/493; G02B 21/368; G02B 21/365; G01N 2015/1006

(57) ABSTRACT

A sediment analysis workstation includes an optical microscope, an image recording device connected to an optical unit of the optical microscope and a user interface device connected to the image recording device. The user interface device comprises one or more processors running computer executable instructions that when executed by the one or more processor causes the one or more processor to count particles of a sediment sample based upon a manual input by an operator, and store counted particles of the sediment sample in a memory of the user interface device. Further, a method and a system for counting particles of a sediment sample are disclosed.

The subject application claims benefit under 35 USC §119(e) of US provisional Application No. 61/984,375, filed Apr. 25, 2014. The entire contents of the above-referenced patent application are hereby expressly incorporated herein by reference.

### FIELD OF INVENTION

A manual sediment analysis workstation, a method for counting sediment particles of a sediment sample and a system for counting particles of a sediment sample are provided. In particular, a manual urine sediment analysis workstation, a method for identifying, characterizing, labeling, and/or counting sediment particles of a urine sample and a system for counting sediment particles of a urine sample are provided.

### BACKGROUND OF INVENTION

In developed regions, urine sediment testing has been automated to a significant extent. These automated analyzers perform all of the functions that would have to be done manually. However, in some countries, for example China, there is a requirement to manually confirm up to 100% of the positive samples flagged by the automated systems.

To analyze urine sediments manually requires a microscope. Operators need to be trained how to recognize and classify the different sediment objects. Training typically requires an experienced operator spending time with one or more trainees. Numerous examples of different ‘positive’ urine sediment samples are needed to enable the trainees to witness the range of specimens they are likely to encounter. Using a microscope for several minutes at a time is fatiguing.

### SUMMARY OF INVENTION

An objective is to provide an improved manual sediment analysis workstation, an improved method for counting sediment particles of a sediment sample, an improved system for counting sediment particles of a sediment sample and an improved method for training operators.

The objectives are achieved with a manual sediment analysis workstation, a method and a system as claimed in the claims.

A sediment analysis workstation, in particular a urine sediment analysis workstation, comprises an optical microscope, an image recording device connected to an optical unit of the optical microscope and a user interface device connected to the image recording device. The user interface device comprises one or more processors running computer executable instructions that when executed by the one or more processor causes the one or more processor to count particles of a sediment sample based upon a manual input by an operator, and to store counted particles of the sediment sample in a memory of the user interface device. The computer executable instructions can be stored in a non-transitory computer-readable storage medium disposed in the user interface or coupled to the user interface.

A method for counting particles of a sediment sample comprises displaying an image of a sediment sample on a screen of a user interface device, and counting particles of the sediment sample using the user interface device based upon manual input by an operator of the user interface device.

A system for counting particles of a sediment sample comprises the sediment analysis workstation with the optical microscope, the image recording device connected to an optical unit of the optical microscope and the user interface device connected to the image recording device. An image of a sediment sample is displayed on a screen of the user interface device, and particles of the sediment sample are counted by the user interface device based upon manual input by an operator of the user interface device.

The sediment analysis workstation, the method for counting particles (also referred to as objects) and the system for counting particles include in particular a urine sediment analysis workstation used for analyzing urine samples.

### BRIEF DESCRIPTION OF THE DRAWINGS

FIG. 1 shows a schematic front view of a manual urine sediment analysis workstation.

FIG. 2 shows a schematic side view of the manual urine sediment analysis workstation of FIG. 1.

FIG. 3 shows a first screen view of a user interface device of the manual urine sediment analysis workstation.

FIG. 4 shows a second screen view of a user interface device of the manual urine sediment analysis workstation.

FIG. 5 shows a third screen view of a user interface device of the manual urine sediment analysis workstation.

FIG. 6 shows a fourth screen view of a user interface device of the manual urine sediment analysis workstation.

FIG. 7 shows a fifth screen view of a user interface device of the manual urine sediment analysis workstation.

FIG. 8 shows a sixth screen view of a user interface device of the manual urine sediment analysis workstation.

### DETAILED DESCRIPTION OF INVENTION

FIG. 1 shows a schematic front view of a manual urine sediment analysis workstation 10.

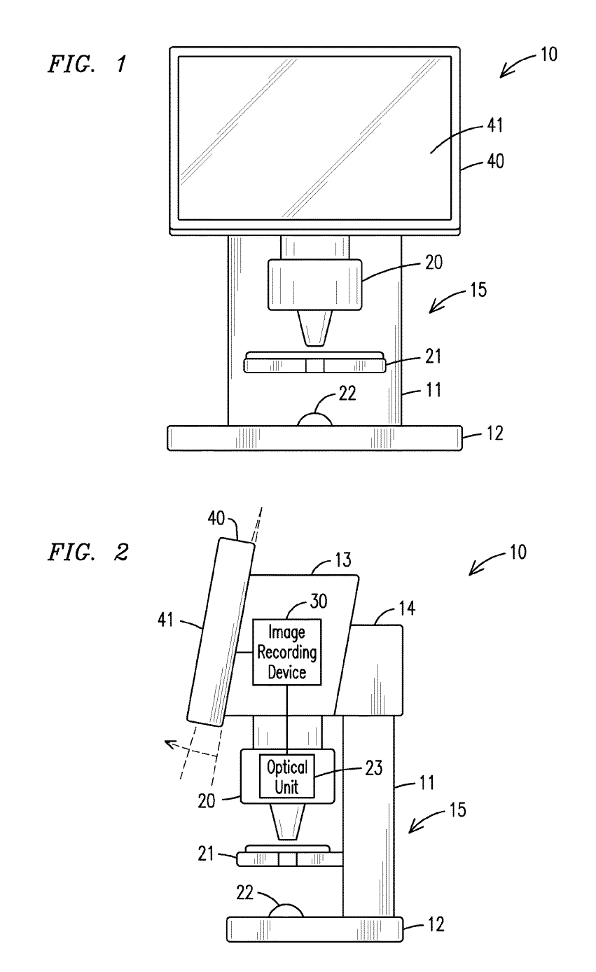

The urine sediment analysis workstation 10 comprises an optical microscope 20, an image recording device 30 (see FIG. 2) connected to an optical unit of the microscope 20, and a user interface device 40 connected to the image recording device 30.

The workstation 10 further comprises a support structure 15, wherein the optical microscope 20, the image recording device 30 and the user interface device 40 are carried by the support structure 15. The support structure 15 includes a stand 11 and a foot 12 which are mechanically connected to each other and carry all the operating equipment for performing the urine sediment analysis. The foot 12 carries the light source 22 for the microscope 20. The stage 21 of the microscope 20 is directly connected to the stand 11 in such a height of the stand 11 that the analysis can be performed properly.

FIG. 1 shows the user interface device 40 including a screen/display 41 which is mechanically connected to the stand 11. The user interface device 40 can be directly or indirectly mechanically connected to the stand 11. According to FIG. 2, the user interface device 40 is mechanically connected to the stand 11 via connecting units 13 and 14.

FIG. 2 shows a schematic side view of the manual urine sediment analysis workstation of FIG. 1.

The workstation 10 comprises an image recording device 30 (only shown schematically) which is connected to the microscope 20, in particular to the optical unit 23 (only shown schematically) of the microscope 20. The image recording device 30 is a camera able to provide video recording. Such a camera is for example a video camera, a digital camera, a cell phone camera or many other cameras which are able to provide video recording. In an embodiment, the camera is integrated into the user interface device 40 providing a compact design and less separate components to be assembled.

The image recording device 30 is electronically connected to the user interface device 40 which is for example a touch screen device with a high-resolution display, e.g. Apple iPad®. However, many other touch screen devices may be used or a custom-made touch screen device for analyzing urine samples is provided.

The user interface device 40 is a computing device and comprises for example one or more processors, one or more memories, a touch screen, one or more inputs and/or outputs, etc. The image recording device 30 is electronically connected (via a cable) to an input of the user interface device 40, wherein an image or an image sequence provided by the microscope 20 via the image recording device 30 is transmitted to the user interface device 40. The image/image sequence is then displayed on the touch screen 41. An image/image sequence will show a urine sample to be manually analyzed by a user with the help of the workstation 10. As indicated by the dotted lines in FIG. 2, the user interface device 40 is attached to the support structure 15, in particular to the connecting unit 13 of the structure 15, so that an angle between the user interface device 40 and the connecting unit 13 is changeable. Therefore, the operator/user can adjust the user interface device 40 if necessary.

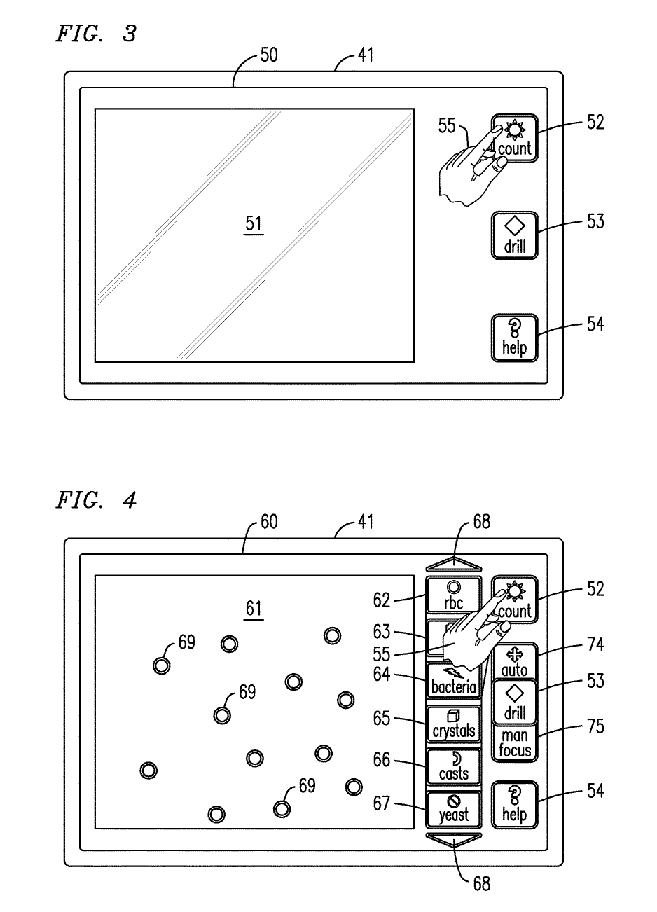

FIG. 3 shows a first screen view 50 of a touch screen 41 of the manual urine sediment analysis workstation 10. The screen view 50 includes an area 51 where a urine sample will be shown (see for example FIG. 4). The touch screen 41 provides several icons, for example icon “count” 52, icon “drill” 53 and icon “help” 54. When one of the icons 52, 53 and 54 is touched on the screen (also called display), as indicated by the hand 55, a command which represents the icon is interpreted and communicated to the appropriate application stored within the touch screen device 40.

The screen view 50 of FIG. 3 shows one embodiment for a set of icons, herein also referred to as main icons. There can be many other icons, which may be for example required according to a specific application or analysis.

The icon 52 “count” is used for identifying, characterizing, labeling, and/or counting different particles/cells/objects of the urine sample. The icon 53 “drill”, when activated, represents a training mode for new users. Such a training mode uses for example pattern recognition algorithms as a learning aid. In another embodiment, sediment recognition algorithms are provided to help the user either with full counting capability, or prompting during training or querying to prevent operator errors. The icon 54 “help” connects to a resource in order to provide the user/operator with information and support related to the urine analysis.

FIG. 4 shows a second screen view 60 of the touch screen 41 of the workstation 10. The view 60 shows the urine sample area 61 comprising sediment 69. Urine sediment usually includes red blood cells, white blood cells, bacteria, crystals, casts, yeast, and miscellaneous (unidentifiable objects).

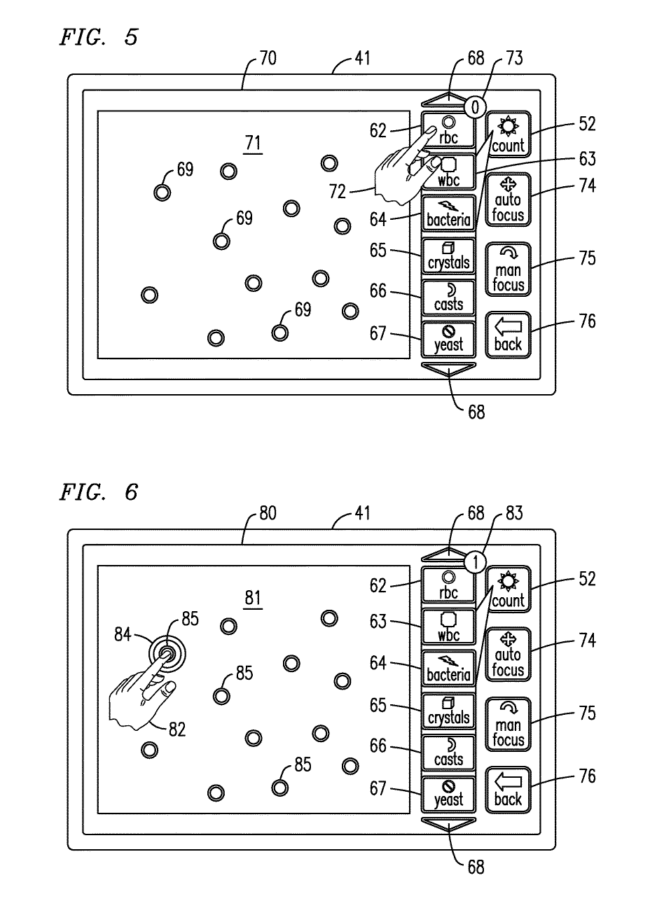

As soon as the user touches the main icon 52 “count”, a further set of icons opens and will be displayed. The set of icons includes icon 62 “rbc” (“red blood cells”), 63 “wbc” (“white blood cells”), 64 “bacteria”, 65 “crystals”, 66 “casts” and 67 “yeast”. As indicated by the arrows 68, further icons can be included, for example “miscellaneous”. FIG. 4 further shows the icons 74 “auto focus” and 75 “man focus” which will be described in connection with FIG. 5.

FIG. 5 shows a third screen view 70 of the touch screen display 41. The urine sample area 71 shows an image of the urine sample transmitted from the microscope via the image recording device (camera) to the touch screen device 40. After activating the icon 52 (see FIG. 4), the user now selects a further icon from the set of icons 62-67 in order to count the sediment of the urine. In the example shown in FIG. 5, the user, indicated by the hand 72, activates the icon 62 in order to count red blood cells in the urine sample. Every icon 62-67 comprises a counter indicating to the user how many cells/particles have been counted. Initially, before the counting has started, the counter 73 of the icon 62 will show “0” as no cells have been counted so far. By doing this the user identifies, characterizes, and labels the sediment so that each type of sediment can be counted and a summary can be created.

The embodiment according to FIG. 5 does not comprise an icon “drill” (as shown in FIG. 3 and FIG. 4), but instead has the icons 74 “auto focus”, 75 “man focus” and 76 “back”. For example, starting from FIG. 3, the user decides to either activate the icon 52 “count” or to enter a training mode by activating the icon 53 “drill”. The user did not activate the icon 53 “drill”, but 52 “count”. Therefore, as indicated in FIG. 4, the new icons 74 and 75 are displayed and the icon 53 disappears. Further, as shown in FIG. 5, the screen view 70 comprises the icon 76 “back”. Although, the icon 54 “help” is not shown in FIG. 5-FIG. 8, the user interface device can be configured such that the icon “help” is displayed at all times. Also, the icon “back” can be displayed at all times (except for the very first screen view as shown in FIG. 3)

FIG. 6 shows a fourth screen view 80 of a touch screen 41. The urine sample area 81 shows the image of the urine sample of FIG. 5. FIG. 6 shows that the user 82 is manually counting the red blood cells 85 by touching the red blood cells 85 on the touch screen 41. When the user touches, i.e. counts, one red blood cell 85, the counter 83 will increase by one. Furthermore, the touch screen device is configured such that a red blood cell 85 which has been counted will be marked as counted. In that way, the user knows which cells have been counted or not. In the example of FIG. 6, a circle 84 is provided around the cell 85. Many other ways can be used to indicate that a cell/particle has been counted, for example strikethrough, cross out, changing the color of the cell, etc. It is important that an indication for already counted cells is provided; otherwise the user may count a cell twice which would lead to false results.

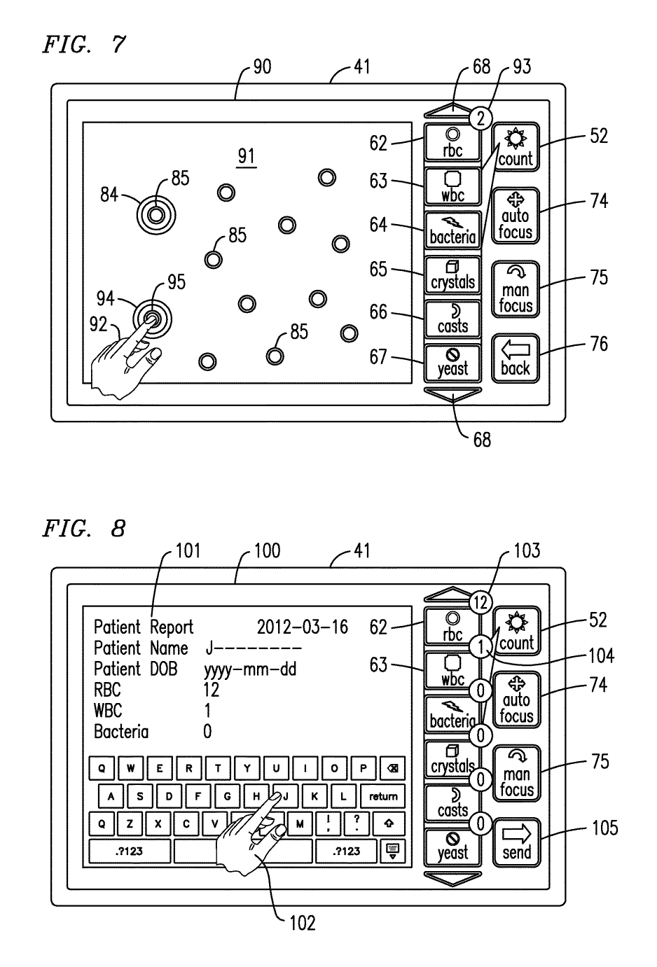

FIG. 7 shows a fifth screen view 90 of a touch screen 41 with the urine sample area 91. FIG. 6 shows that the first red blood cell 85 has been counted. In FIG. 7, a second red blood cell 95 is manually counted by the user 92, indicated by the circle 94. Also, the counter 93 of the icon 62 shows the number of red blood cells counted.

In an analogous manner, the user will now count one or more other types of cells/particles in the urine sample, for example white blood cells, bacteria, crystals, etc.

FIG. 8 shows a sixth screen view 100 of a touch screen 41 of the touch screen device 40 of the manual urine sediment analysis workstation 10. When counting of the urine sediment has been completed, an electronic patient report 101 is automatically displayed. The user 102 is required to provide the name and the date of birth of the patient to whom the urine sample belongs to so that the count of the urine sediment is properly stored and retrievable by name of the patient. The electronic patient report 101 is dated. The input of the date of birth of the patient can be optional.

As can be seen on the right side of the screen view 100, the icons 62-67 indicate by their corresponding counters how many cells/particles have been counted. For example, the counter 103 shows that twelve red blood cells have been counted. The counter 104 shows that one white blood cell has been counted. This data is automatically stored in the electronic patient report 101 so that the user 102 does not have to type in this information. The electronic patient report 101 as shown in FIG. 8 is only one embodiment. In another embodiment, the patient report may include other fields which have to be manually filled in, for example number of urine sample (first or second sample counted), or which are provided automatically, for example a time stamp when the urine analysis has been completed.

FIG. 8 further shows the icon 105 “send” which can be used to send the electronic patient report 101 to a Laboratory Information System (LIS) or a data manager or to an external data base. In an embodiment, the interface user device therefore comprises a network port and/or Wi-Fi for sending the patient report and/or image data and/or counting data of the urine sample to the LIS or any other data base.

In a way, a semi-automated urine sediment analysis workstation is provided. While the particles/cells of the urine sample are visually and manually identified by the operator, the workstation automatically counts the particles/cells identified by the operator and stores these data in a memory of the user interface device.

Summarizing, the method for counting particles of a sediment sample includes displaying an image of a sediment sample on a screen of a user interface device, and counting particles of the sediment sample using the user interface device based upon manual input by an operator of the user interface device. The user interface device is a touch screen device, wherein the operator visually identifies the particles and touches visually identified particles on the touch screen. The user interface device comprises one or more processors running computer executable instructions that when executed by the one or more processor causes the one or more processor to count the particles of the sediment sample when the particles are touched by the operator on the touch screen, and to store counted particles of the sediment sample in a memory of the user interface device. The computer executable instructions can be stored in a non-transitory computer-readable storage medium disposed in the user interface or coupled to the user interface. The user interface device is configured such that different types of particles are countable, and wherein the different types of particles are stored separately after counting. For every type of particles an icon is displayed on the touch screen and, before counting a type of particles, the corresponding icon is activated. An electronic patient file is displayed on the screen after counting of the particles has been completed.

The workstation is embodied as a compact device (“one-box” device) incorporating all the equipment for performing the analysis of the sediment samples so that no extra equipment is necessary. The user interface is specifically designed to make counting objects easier and less fatiguing for novice users.

In an embodiment, one or more images of the urine sample are stored internally in the user interface device or on a removable storage medium, for example a compact disc, a USB stick, etc. The one or more images of the urine samples may be stored as jpegs which then can be recalled to the touch screen.

In another embodiment, a color temperature of illumination LED of the sediment sample is changeable for a better image contrast.

In another embodiment, the sediment count, patient data and operator identification (ID) are added as tags in the image metadata. In this case, the patient data and operator ID are provided automatically (instead of manually). The workstation is not only used for the urine analysis but doubles as a data entry terminal.

In another embodiment, the interface user device, for example the touch screen device, comprises multiple USB ports to connect alternate user input devices, for example keyboard and mouse, if preferred to the touchscreen.

In another embodiment, a barcode reader function is provided. For example, the work station comprises a barcode reader and every urine sample is provided with a barcode. When analyzing the urine sample, the barcode reader of the work station, which is for example integrated into the user interface device, reads to barcode which includes for example information with regard to patient identification (for example name and date of birth) and operator identification. The patient demographics and/or operator ID are then automatically loaded into the electronic patient report. In this embodiment, work flow steps and error sources are reduced when the patient demographics and/or operator ID are loaded automatically instead of typed in manually.

The following examples are provided for illustrative purposes only and are intended to be non-limiting examples only.

In an first illustrative example, a sediment analysis workstation comprises: (1) an optical microscope, (2) an image recording device connected to an optical unit of the optical microscope, and (3) a user interface device connected to the image recording device, wherein the user interface device comprises one or more processors running computer executable instructions that when executed by the one or more processor causes the one or more processor to count particles of a sediment sample based upon a manual input by an operator, and store counted particles of the sediment sample in a memory of the user interface device. The computer executable instructions can be stored in a non-transitory computer-readable storage medium disposed in the user interface or coupled to the user interface. In a second illustrative example, the user interface is capable of training operators by employing and displaying a number of pre-classified sediment sample images. In a third illustrative example, the user interface device is a touch screen device. In a fourth illustrative example, the image recording device is a camera including video recording function. In a fifth illustrative example, the camera is integrated into the user interface device. In a sixth illustrative example, the workstation further comprises a support stand, wherein the optical microscope, the image recording device and the user interface device are carried by the support stand.

A first illustrative method for counting particles of a sediment sample comprises displaying an image of a sediment sample on a screen of a user interface device, and counting particles of the sediment sample using the user interface device based upon manual input by an operator of the user interface device. In a second illustrative method, the user interface device is a touch screen device and wherein the operator visually identifies the particles and touches visually identified particles on the touch screen. In a third illustrative method, the user interface device comprises one or more processors running computer executable instructions that when executed by the one or more processor causes the one or more processor to: count the particles of the sediment sample when the particles are touched by the operator on the touch screen, and store counted particles of the sediment sample in a memory of the user interface device. A fourth illustrative method comprises: connecting an image recording device to an optical microscope and a user interface device to the image recording device, and transmitting an image of the sediment sample provided by the microscope and recorded by the image recording device to the user interface device. In a fifth illustrative method, the user interface device is configured such that different types of particles are countable, and wherein the different types of particles are stored separately after counting. In a sixth illustrative method, the user interface device is configured such that for every type of particles an icon is displayed on the touch screen and, before counting a type of particles, the corresponding icon is activated. In a seventh illustrative method, the user interface device is configured such that an electronic patient file is displayed on the screen after counting of the particles has been completed.

A first illustrative system for counting particles of a sediment sample comprises: a sediment analysis workstation comprising: an optical microscope, an image recording device connected to an optical unit of the optical microscope, a user interface device connected to the image recording device, wherein an image of a sediment sample is displayed on a screen of the user interface device, and wherein particles of the sediment sample are counted by the user interface device based upon manual input by an operator of the user interface device. In a second illustrative system, the user interface device comprises one or more processors running computer executable instructions that when executed by the one or more processor causes the one or more processor to: count particles of a sediment sample based upon a manual input by an operator, and store counted particles of the sediment sample in a memory of the user interface device. In a third illustrative system the user interface device is configured such that different types of particles are countable, and wherein the different types of particles are stored separately after counting. In a fourth illustrative system the user interface device is configured such that for every type of particles an icon is displayed on the touch screen and, before counting a type of particles, the corresponding icon is activated. In a fifth illustrative system, the user interface device is configured such that an electronic patient file is displayed on the screen after counting of the particles has been completed. In a sixth illustrative system, the user interface device is a touch screen device. In a seventh illustrative system the image recording device is a camera including video recording function. In an eighth illustrative system, the system further comprises a support stand, wherein the optical microscope, the image recording device and the user interface device are carried by the support stand.

While specific embodiments have been described in detail, those with ordinary skill in the art will appreciate that various modifications and alternative to those details could be developed in light of the overall teachings of the disclosure. Accordingly, the particular arrangements disclosed are meant to be illustrative only and not limiting as to the scope of the invention, which is to be given the full breadth of the appended claims, and any and all equivalents thereof

### LIST OF REFERENCE NUMBERS

10—Workstation
11—Stand
12—Foot
13—Connecting Unit
14—Connecting Unit
15—Support Structure
20—Microscope
21—Stage
22—Light Source
23—Optical Unit
30—Image Recording Device
40—User Interface Device
41—Screen/Display
50—First Screen View
51—Sample Area
52—Icon “count”
53—Icon “drill”
54—Icon “help”
55—User/Operator
60—Second Screen View
61—Sample Area
62—Icon “rbc” (Red Blood Cells)
63—Icon “wbc” (White Blood Cells)
64—Icon “bacteria”
65—Icon “crystals”
66—Icon “casts”
67—Icon “yeast”
68—Arrows
69—Sediment
70—Third Screen View
71—Sample Area
72—User/Operator
73—Counter
74—Icon “auto focus”
75—Icon “man focus”
76—Icon “back”
80—Fourth Screen View
81—Sample Area
82—User/Operator
83—Counter
84—Circle
85—Red Blood Cells
90—Fifth Screen View
91—Sample Area
92—User/Operator
93—Counter
94—Circle
95—Red Blood Cell
100—Sixth Screen View
101—Electronic Patient Report
102—User/Operator
103—Counter
104—Counter
105—Icon “send”

What is claimed is:

1. Sediment analysis workstation comprising:
an optical microscope,
an image recording device connected to an optical unit of the optical microscope,
a user interface device connected to the image recording device,
wherein the user interface device comprises one or more processors running computer executable instructions that when executed by the one or more processor causes the one or more processor to
count particles of a sediment sample based upon a manual input by an operator, and
store counted particles of the sediment sample in a memory of the user interface device.

2. The workstation as claimed in claim 1, wherein the user interface device is capable of training operators by employing and displaying a number of pre-classified sediment sample images

3. The workstation as claimed in claim 1, further comprising a support stand, wherein the optical microscope, the image recording device and the user interface device are carried by the support stand.

4. A method for counting particles of a sediment sample comprising:
displaying an image of a sediment sample on a screen of a user interface device, and
counting particles of the sediment sample using the user interface device based upon manual input by an operator of the user interface device.

5. The method as claimed in claim 4, wherein the user interface device is a touch screen device and wherein the operator visually identifies the particles and touches visually identified particles on the touch screen.

6. The method as claimed in claim 5, wherein the user interface device comprises one or more processors running computer executable instructions that when executed by the one or more processor causes the one or more processor to
count the particles of the sediment sample when the particles are touched by the operator on the touch screen,
store counted particles of the sediment sample in a memory of the user interface device.

7. The method as claimed in claim 4, further comprising:
connecting an image recording device to an optical microscope and a user interface device to the image recording device, and
transmitting an image of the sediment sample provided by the microscope and recorded by the image recording device to the user interface device.

8. The method as claimed in claim 6, wherein the user interface device is configured such that different types of particles are countable, and wherein the different types of particles are stored separately after counting.

9. The method as claimed in claim 8, wherein the user interface device is configured such that for every type of particles an icon is displayed on the touch screen and, before counting a type of particles, the corresponding icon is activated.

10. The method as claimed in claim 4, wherein the user interface device is configured such that an electronic patient file is displayed on the screen after counting of the particles has been completed.

11. A system for counting particles of a sediment sample comprising:
a sediment analysis workstation comprising:
an optical microscope,
an image recording device connected to an optical unit of the optical microscope,
a user interface device connected to the image recording device,
wherein an image of a sediment sample is displayed on a screen of the user interface device, and
wherein particles of the sediment sample are counted by the user interface device based upon manual input by an operator of the user interface device.

12. The system as claimed in claim 11, wherein the user interface device comprises one or more processors running computer executable instructions that when executed by the one or more processor causes the one or more processor to
count particles of a sediment sample based upon a manual input by an operator, and
store counted particles of the sediment sample in a memory of the user interface device.

13. The system as claimed in claim 12, wherein the user interface device is configured such that different types of particles are countable, and wherein the different types of particles are stored separately after counting.

14. The method as claimed in claim 13, wherein the user interface device is configured such that for every type of particles an icon is displayed on the touch screen and, before counting a type of particles, the corresponding icon is activated.

15. The system as claimed in claim 11, wherein the user interface device is configured such that an electronic patient file is displayed on the screen after counting of the particles has been completed.
